(19) United States
(12) Patent Application Publication
(10) Pub. No.: US 2013/0150840 A1
(43) Pub. Date: Jun. 13, 2013

(54) SURGICAL MICROSCOPE SYSTEM FOR OPHTHALMOLOGY, AND ASSOCIATED DETECTION UNIT

(71) Applicant: Leica Microsystems (Schweiz) AG, Heerbrugg (CH)

(72) Inventor: Ulrich Sander, Rebstein (CH)

(73) Assignee: LEICA MICROSYSTEMS (SCHWEIZ) AG, Heerbrugg (CH)

(21) Appl. No.: 13/708,002

(22) Filed: Dec. 7, 2012

(30) Foreign Application Priority Data
Dec. 8, 2011 (DE) 10 2011 088 038.0

(51) Int. Cl. A61B 3/13; A61F 9/008

(52) U.S. Cl. CPC A61B 3/13; A61F 9/008
USPC 606/6; 351/221; 351/215

(57) ABSTRACT

A surgical microscope system (100) for ophthalmology, in particular for cataract surgery, is proposed, which comprises an illumination unit (10) and a viewing unit (30), the surgical microscope system (100) being set up to irradiate illumination light (15) of the illumination unit (10) into an eye (50) arranged on the objective side of the surgical microscope, the surgical microscope system (100) comprising a detection unit (20) by means of which a scattering (15′) of the illumination light (15) irradiated into the eye (50) is determinable, in the form of a scattering pattern and/or scattering coefficient, by sensing a portion of the illumination light (15) radiated back by the eye (50).

### CROSS REFERENCE TO RELATED APPLICATIONS

This application claims priority of German patent application number 10 2011 088 038.0 filed Dec. 8, 2011, the entire disclosure of which is incorporated by reference herein.

### FIELD OF THE INVENTION

The present invention relates to a surgical microscope system for ophthalmology, in particular for the investigation and/or surgical treatment of a cataract of an eye, and to a detection unit for a surgical microscope system of this kind

### BACKGROUND OF THE INVENTION

The term “cataract” refers to a turbidity of the lens of the eye, i.e. a decrease in its transparency. Certain forms of cataracts develop relatively quickly, but the great majority develop over a period of several decades. Severe forms of cataracts therefore occur principally in older people. Surgical methods for the removal of cataracts have been known for some time, and represent routine procedures. The lens is usually replaced in this context by a plastic lens (intraocular lens, IOL).

In almost all forms of cataracts, the impairment in vision is based on a structural change in the lens that results in turbidity.

Cataract surgery is not entirely risk-free. Because cataracts develop slowly, the question as to the correct time for an operation is therefore in some circumstances difficult to answer.

For stability reasons, the posterior capsule of the lens of the eye is not removed in the cataract procedure. Serious complications can otherwise occur. On the other hand, fibrous proliferation of certain cells (“capsular fibrosis”) can occur on the remaining posterior lens capsule after the operation, causing turbidity to reoccur. Visual impairments can also, however, arise principally because lens residues or very thin membranes remain behind on the posterior lens capsule. An “after-cataract” of this kind forms in up to 30% of cases after a cataract operation. The exact causes are not known; this is also due to an absence of objective measurement methods, for example to check surgical success. Further operations or laser treatments are necessary in order to remove the after-cataract.

No capability so far exists for intra- or post-operatively assessing and quantifying the state of the posterior lens capsule during the operation. Maximally complete removal of lens residues, membranes, etc., also called “lens polishing,” is usually performed exclusively visually, and can therefore lead to the aforementioned residual risk.

In light of this, a need therefore exists for improved surgical microscope systems that offer corresponding diagnostic capabilities.

### SUMMARY OF THE INVENTION

The present invention provides a surgical microscope system for ophthalmology, in particular for the investigation and/or surgical treatment of a cataract of an eye, as well as a detection unit for a surgical microscope system of this kind The surgical microscope system comprises an illumination unit and a viewing unit. The surgical microscope system is set up to irradiate illumination light of the illumination unit into an eye arranged on the objective side of the surgical microscope. The surgical microscope system also comprises a detection unit by means of which a scattering of the illumination light irradiated into the eye is determinable, in the form of a scattering pattern and/or a scattering coefficient, by sensing a portion of the illumination light radiated back by the eye.

The present invention proceeds from a known surgical microscope having an illumination unit. Illumination light can be irradiated by the illumination unit into a patient's eye arranged on the objective side of the surgical microscope. The surgical microscope comprises a viewing unit of known type, by means of which a microscopic image of the eye can be viewed by the surgeon.

According to the present invention, the surgical microscope additionally comprises a detection unit. The detection unit makes it possible to determine a scattering of the illumination light irradiated into the eye. This is done by sensing, in the form of a scattering pattern and/or scattering coefficient, a portion of the illumination light radiated back by the eye.

The scattering pattern is preferably a two- or three-dimensional representation of the scattering structures in the eye being investigated. Advantageously, this scattering pattern corresponds in its dimensions, at least in part, to the observed optical microscopic image. It can therefore be sensed and/or viewed together with the latter, for example in the form of an injected image or superimposition. Scattering structures in the observed eye, e.g. membrane residues on the posterior lens capsule, are more easily recognized as a result.

Alternatively or additionally, a scattering coefficient can be determined by the detection unit. A numerical scattering coefficient that is correlated with the proportion of scattering structures in the observed eye can thus, for example, be indicated in addition to or instead of the scattering pattern. For example, the scattering coefficient represents the areal proportion of the scattering structures in relation to the total observed area. Another possibility would be to provide an information item (number, color, text) which indicates to the surgeon whether membrane residues have been sufficiently removed, or whether further lens polishing is necessary.

The detection device makes it possible to quantitatively sense residual turbidity due to membrane residues intra- and post-operatively in the course of a cataract operation, and to take corresponding surgical actions. For example, a decision can be made as to whether “lens polishing” is to be continued or terminated. This makes possible complete elimination of disruptive scattered-light sources, for example on the posterior lens capsule.

An advantageous detection method can correspond at least in part to the one disclosed in WO 03/009745 A2. In this, polarized light is irradiated through the lens onto the retina. A portion of the light is reflected by the retina. An image of the front side of the lens can be generated via an imaging optic, and acquired by a camera. The extent of light scattering can be sensed in a detection unit.

By means of the surgical microscope system according to the present invention, illumination light can also be irradiated through the lens of the eye, or through corresponding lens structures that have not been removed, into an eye. The light is focused by the lens body, if it is still present. The illumination light is reflected by the retina and thereby at least in part radiated back toward a detection unit. The method can be used even if the lens body has already been removed. In this case the illumination light is not focused onto the retina, but is nevertheless (diffusely) reflected by it. Scattering structures are detectable in this way as well.

The illumination light is scattered at existing turbidity points both when irradiated into the eye and after reflection at the retina. Scattering upon irradiation into the eye is also visible with the naked eye in the form of a grayish to whitish clouding of the lens of the eye (“gray cataract”). The illumination light is, however, also correspondingly scattered after reflection. This causes a perceptible attenuation of the reflected light at the scattering structures. The aforesaid scattering effects are also referred to in the context of this Application as the “first” and the “second” scattering.

Both the first and the second scattering have diagnostic value, and permit conclusions as to the presence of turbidity. The diagnostic value can be further intensified by irradiating light of (a) specific wavelength(s) and/or polarization(s).

The totality of the light “back-radiated” from the object therefore contains portions from the first scattering and, correspondingly attenuated, from the reflection from the retina.

It can be advantageous to use illumination light having a first polarization extent, and to detect with the detection unit light having a second polarization extent. The illumination unit and detection unit are correspondingly set up for this.

A “polarization extent” can be understood as a specific polarization angle, but also as a range of polarization angles of partly polarized light. Completely depolarized light accordingly also possesses a polarization extent, which covers the entire range of polarization angles.

For example, linearly polarized light having a first polarization angle can be irradiated into the eye. When polarized light is reflected at reflective surfaces, for example at the surface of the cornea in the eye, its polarity is maintained. Conversely, when light is reflected from a matte surface, for example from the retina or from the scattering structures in the eye, its polarization is lost. If a blocking filter for the first polarization angle is provided in front of or in the detection device, light reflected at reflective surfaces can be simply and effectively blocked out. The result is that only the light reflected at the matte surface of the retina, or in the context of the first scattering, is sensed. Reflections at the cornea are no longer apparent as interference.

In order to achieve the aforesaid advantages, a surgical microscope system comprises at least two polarization devices. For example, a first linear polarizer can be associated with the illumination unit, and a second linear polarizer with the detection unit.

As explained in further detail below, both polarity-specific and wavelength-specific illumination and/or evaluation can offer particular diagnostic advantages.

In order to sense the illumination light radiated back from the eye, a corresponding detection unit comprises a first beam splitter. This couples out at least a portion of the illumination light radiated back by the eye. Defined, diagnostically valuable portions of the back-radiated light can be respectively outcoupled by appropriately selecting the beam splitter, for example in the form of a semitransparent mirror. For turbidity detection, for example, illumination light having wavelengths in the non-visible region, e.g. infrared light, can be used. Visual observation by the surgeon during the operation is thus not impaired. A corresponding wavelength region can be selectively outcoupled and diagnostically utilized.

A scattering pattern having diagnostic value can be obtained from both the first scattering and the second scattering. In the case of planar irradiation of illumination light, i.e. spreading of a corresponding beam bundle, a planar scattering pattern corresponding to the scattering structures is obtained. First and second scattering patterns can also be correlated with or offset from one another, with the result that an additionally improved assessment of turbidity can be accomplished.

In order to determine the scattering pattern and/or scattering coefficient, the detection unit advantageously comprises an optical unit, an image acquisition device, and/or a calculation unit. A scattering pattern that is obtained can be processed in this context, for example using corresponding image processing software. Entirely optical processing of the scattering pattern is also possible.

The detection unit advantageously comprises at least one output for outputting the scattering pattern or scattering coefficient. The scattering pattern can be read out in any way, or further processed in particular to yield the aforesaid scattering coefficient. This makes possible, for example, presentation on a screen, transfer via a network, and/or recording for documentation purposes. The output can occur digitally, in analog fashion, and/or in the form of an optical recording.

An advantageous viewing unit of a surgical microscope system possesses an superimposition device coupled to at least one of the outputs of the detection device. By means of the superimposition device, the scattering pattern or scattering coefficient can be electronically or optically superimposed onto a microscopic object image. The surgeon can thereby make a continuous assessment of the success of the procedure. Advantageously, a superimposition device of this kind is embodied to be switchable in and out, so that viewing of an object without a superimposition is also possible.

It is regarded as advantageous, in a corresponding surgical microscope system, to couple the illumination light by means of a beam splitter into the observation beam path of the surgical microscope. An illumination beam path can thereby be brought into congruence with an observation beam path, thereby ensuring good matching between the scattering pattern and the object image.

Advantageously, the illumination unit generates illumination light of a specific wavelength extent, in particular infrared light. Light of a specific wavelength can also be used.

It is known that when illuminated with white light, the retina reflects in the red spectral region and the macula in the yellow spectral region. A differential evaluation of the first and the second scattering can therefore be accomplished using suitable wavelengths. For example, if blue light is irradiated into the eye, it is reflected very little or not at all by the retina. The light components reflected back from the eye are therefore to be attributed predominantly to the first scattering.

Particular advantages can also be achieved if the previously explained polarization-specific illumination and/or sensing is combined with a wavelength-specific illumination and/or sensing of this kind For example, infrared light that additionally has a defined polarization angle can be irradiated into the eye, and only depolarized infrared light can be sensed in the detection device. Image features or image data correspondingly obtained can be selectively and deactivatably coupled into a visual image in a superimposition device. Scattering structures can also, for example, be presented in intensified fashion or in false color in the superimposed image. The overall result is to implement a diagnostically very valuable auxiliary illumination that does not influence visual observation by the surgeon.

Advantageously, a treatment unit is associated with the surgical microscope system. This unit can encompass, for example, a short-pulse laser, such as a nanosecond or femtosecond laser. A cataract-affected lens can be easily, in some cases automatically, and effectively treated or disintegrated using a laser beam of this kind.

As mentioned, a corresponding surgical microscope system is particularly suitable for ophthalmology, in particular for cataract surgery.

Regarding the detection unit that is likewise proposed according to the present invention, reference is expressly made to the advantages and features explained above.

Further advantages and embodiments of the invention are evident from the description and the appended drawings.

It is understood that the features recited above and those yet to be explained below are usable not only in the respective combination indicated, but also in other combinations or in isolation, without departing from the scope of the present invention.

### BRIEF DESCRIPTION OF THE DRAWING VIEWS

The invention is schematically depicted in the drawings on the basis of an exemplifying embodiment, and will be described in detail below with reference to the drawings.

FIG. 1 schematically depicts a surgical microscope system according to a particularly preferred embodiment of the invention.

FIG. 2 is a detailed depiction of a surgical microscope system according to a particularly preferred embodiment of the invention.

FIGS. 3a and 3b schematically depict the manner of operation of a surgical microscope system according to a particularly preferred embodiment of the invention.

FIGS. 4a and 4b schematically depict the manner of operation of a surgical microscope system according to a particularly preferred embodiment of the invention.

FIG. 5 is an image obtainable by means of a detection unit according to a particularly preferred embodiment of the invention.

### DETAILED DESCRIPTION OF THE INVENTION

In the Figures that follow, identical elements are indicated using identical reference characters and, for the sake of clarity, are not explained repeatedly.

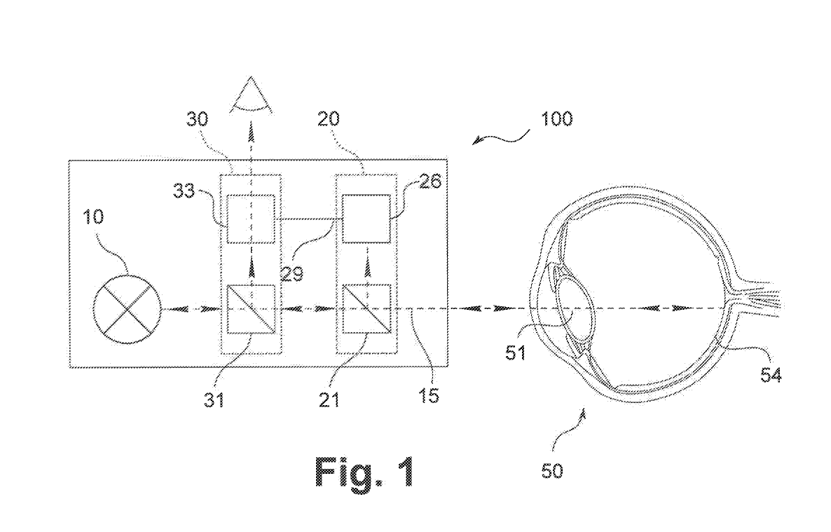

In FIG. 1 a surgical microscope system with its essential components, according to a particularly preferred embodiment of the invention, is schematically depicted and labeled 100 in its entirety.

Surgical microscope system 100 encompasses an illumination unit 10. Illumination light 15, as illustrated by the dashed beam path of FIG. 1, can be irradiated by illumination unit 10 into an eye 50 of a patient. Surgical microscope system 100 further encompasses at least one viewing unit 30 by means of which a microscopic image of eye 50 can be viewed. A detection unit 20, which is set up to sense illumination light 15 radiated back through eye 50, is provided. A beam splitter 21 is associated with detection unit 20, and a beam splitter 31 is associated with viewing unit 30.

Illumination light 15 irradiated by illumination unit 10 can be, in particular, polarized light such as that which can be generated and/or selected by the use of suitable light sources and/or polarizing filters. Illumination unit 10 can preferably also comprise an infrared light source, for example a diode emitting infrared light (IR LED). The beam path of illumination light 15 passes through beam splitter 31 associated with viewing unit 30 and through beam splitter 21 associated with detection unit 20. Corresponding illumination light 15 is irradiated into eye 50 when the microscope is used.

In eye 50, illumination light 15 shines through the optical media of the eye and arrives at retina 54. Illumination light 15 is at least partly reflected by retina 54. The reflected illumination light shines at least in part again through the optical media of eye 50, and can be sensed in surgical microscope system 100 as a back-radiated component of illumination light 15.

The back-radiated illumination light 15 is at least partly outcoupled in beam splitter 21 associated with detection unit 20. In a detection unit 20, of which only a calculation unit 26 is depicted in FIG. 1, a scattering pattern that represents the scattering in eye 50 can be generated from the outcoupled component. Corresponding scattering patterns, e.g. in the form of processed image data, can be conveyed via an output 29 of calculation unit 26 to a superimposition device 33 of a viewing unit 30, and superimposed therein onto a microscopic image that has been obtained. Alternatively or additionally, the previously discussed scattering coefficient can be made available to the surgeon, for example once again by superimposition onto a specific location on the microscopic image. The scattering coefficient can, for example, be calculated from the scattering pattern. It refers, for example, to the areal proportion of the scattering structures in relation to the total area being investigated. An indication in color can also reproduce the scattering coefficient, for example in the form of a “traffic light” (red: scattering coefficient too high; further lens polishing required—yellow: further membrane residues should be removed—green: proportion of scattering structures is below the acceptable limit).

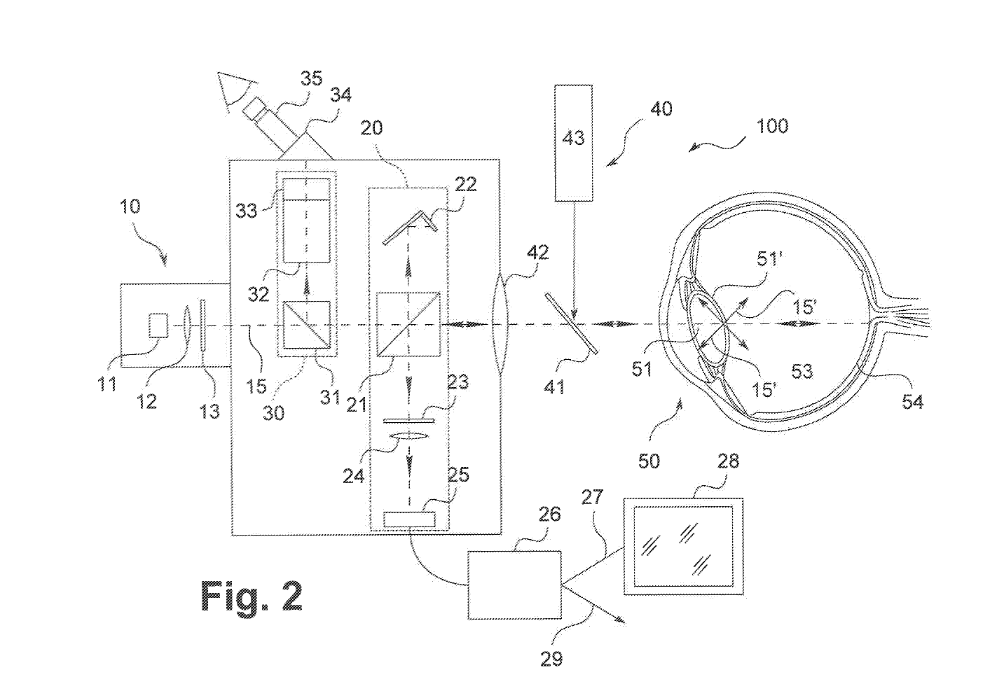

FIG. 2 depicts in detail a surgical microscope system 100 that corresponds to the basic concept explained in FIG. 1. As illustrated schematically here, illumination unit 10 encompasses, for example, a light source 11 in the form of an IR

LED and/or a light source that emits light in the red spectral region. A so-called “red reflex” illumination can be realized using illumination unit 10. This is already implemented in many surgical microscopes for ophthalmology.

Because the light reflected at the retina exhibits intensity maxima in the red spectral region, the term “red reflex” is used. The smaller the illumination angle, the more pronounced this red reflex becomes; as a rule, the illumination beam path is guided via the main objective of the surgical microscope, and the axis of the main objective serves as a reference axis for the illumination angle. Illumination angles in the range between −2° and +2° are suitable for good red reflex illumination.

Light source 11 has associated with it, for example, an optical system, made up in this case of a lens 12 and a filter 13. By means of the optical system, a collimated illumination light 15 having specific optical properties, for example having a specific polarization extent or a specific wavelength range, can be selected. Filter 13 is preferably a linear polarizer that selects linearly polarized light.

Beam splitter 31 associated with viewing unit 30 is preferably set up to selectively allow the passage of light that is selected by filter 13, for example light of a specific polarization, but to outcouple light having other properties.

Arranged in the beam path of illumination light 15 after beam splitter 31 is a beam splitter 21 that is associated with detection unit 20. The light outcoupled by beam splitter 21 is directed into a light trap 22. A corresponding light trap 22 can also be associated with beam splitter 31.

A further optical unit 42, for example an objective, is provided after beam splitter 21 in the beam path of illumination light 15.

A further optical element 41, for example a further filter, mirror, or polarizer, is provided on the object side of surgical microscope system 100. The beam path of the illumination light passes through the elements explained above, and into an eye 50.

A lens 51 of the eye, a posterior lens capsule 51′, a vitreous body 53, and a retina 54 are depicted as anatomical structures or optical media of eye 50. If lens 51 has been removed in the context of a cataract operation, but if lens residues (membranes) are still present on posterior lens capsule 51′, they cause scattering of illumination light 15. This is illustrated by arrows 15′. A first scattering is caused when illumination light 15 enters the eye, and a second scattering after reflection at retina 54. Light radiated back toward surgical microscope system 100 shines through further optical element 41 and objective 42, and is sensed in surgical microscope system 100.

At least a portion of the back-radiated light is outcoupled at beam splitter 21. A further optical system, e.g. having a filter 23 and a collimator lens 24, is provided, and images the outcoupled light onto a detection device 25, for example a CCD chip or a digital camera. Signals of detection device 25 are evaluated by means of a calculation unit 26. Evaluated data can be outputted via an output 27 to a display system 28 and displayed there. The evaluated data can be conveyed via a further output 29, for example, to superimposition device 33 that is associated with viewing unit 30.

As explained, viewing unit 30 encompasses a beam splitter 31. Also provided is a zoom system 32 with which the magnification of a microscopic image can be modified. Located after the zoom system is superimposition device 33. With the latter, the image outputted via output 29 of calculation unit 26 can be superimposed onto the microscopic image in the form of a scattering pattern.

A tube 34 and an eyepiece 35 are depicted merely schematically. Surgical microscope system 100 is preferably a stereomicroscope, so that the optical components of surgical microscope system 100 are provided at least in part in double fashion in order to obtain a stereoscopic image of eye 50 being observed.

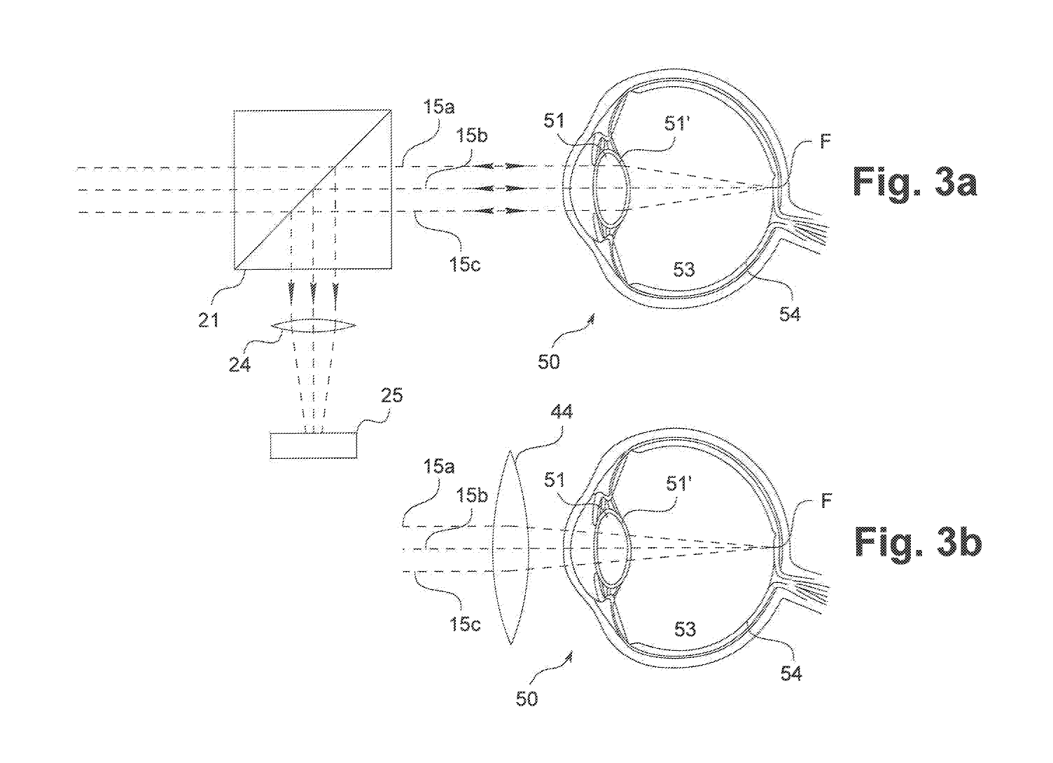

The manner of operation of detection unit 20 will be explained with reference to FIGS. 3a and 3b. As explained previously, a collimated light bundle is irradiated into an eye 50 by means of an illumination unit 10. The collimated light bundle is schematically illustrated in FIGS. 3a and 3b by light beams 15a, 15b, and 15c. FIG. 3a shows beam splitter 21 of detection unit 20, by means of which back-radiated light can be partly outcoupled.

FIG. 3a shows an eye with the lens body of lens 51 still present. In FIG. 3b the lens body is assumed to have been removed, so that lens 51 has no, or only very little, refractive power. In this case an additional lens 44, for example a 15-diopter lens, can be used. By means of this, instead of the removed lens, collimated illumination light 15a, b, c can be focused onto retina 54. Although FIGS. 3a and 3b each depict focusing of the illumination light onto a focal point F on the retina, it is to be understood that the method according to the present invention does not necessarily require focusing.

Illumination light 15a, b, c is reflected by retina 54 of eye 50 and thereby in part radiated back toward surgical microscope system 100 or its beam splitter 21. A portion of the light is coupled out at beam splitter 21 and imaged, for example, by means of a lens 24 onto a detection device 25.

FIGS. 3a and 3b illustrate a situation in which substantially no scattering of the irradiated illumination light 15a, b, c is taking place. Light irradiated into eye 50 is therefore radiated back substantially completely, aside from reflection losses at retina 54, into beam splitter 21. This situation corresponds to that of a healthy eye (FIG. 3a) or to that of an eye that has been operated on and in which turbidity on the posterior capsule has been completely removed.

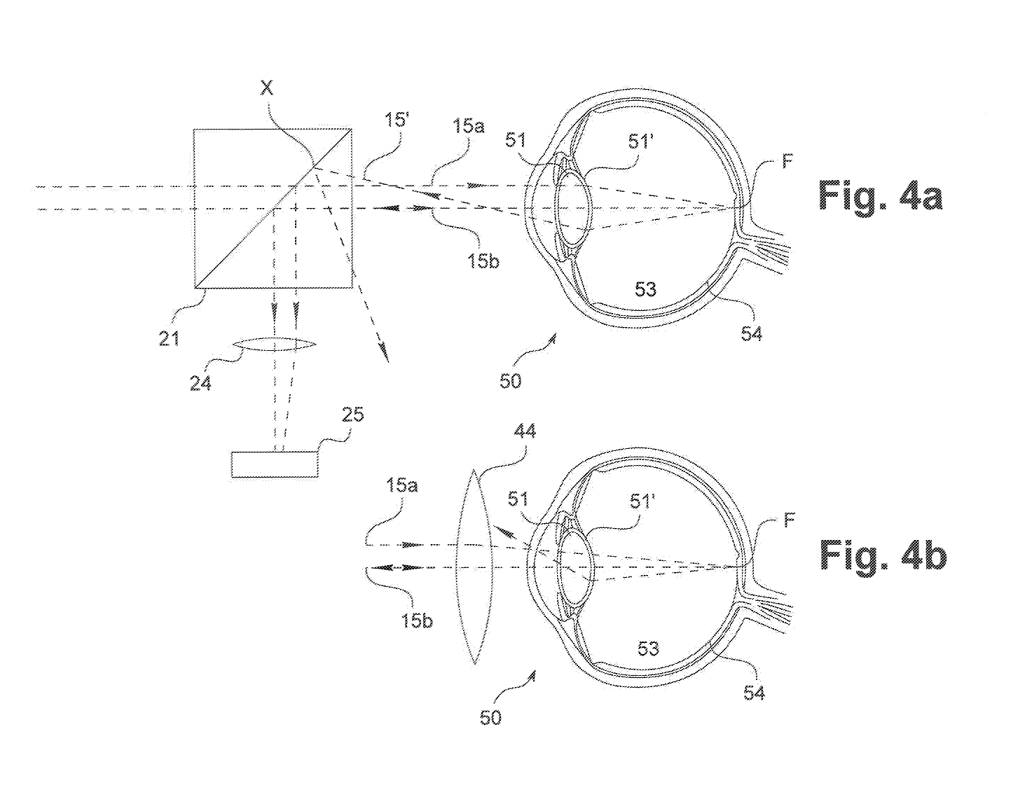

FIGS. 4a and 4b, which otherwise correspond to FIGS. 3a and 3b, illustrate a situation in which membrane residues are present in a lens 51 or on posterior lens capsule 51′ and cause scattering. FIGS. 4a and 4b illustrate only the second scattering after reflection at the retina. Only two light beams 15a and 15b are depicted. Light beam 15b shines through those anatomical elements or optical media of lens 51 which are still present, and is reflected at a point F on retina 54. The reflected light beam 15b is not scattered at the anatomical elements of lens 51 that are present. As above, outcoupling occurs at beam splitter 21.

Light beam 15a likewise shines through lens 51 and is reflected at point F of retina 54. The reflected light beam 15a is, however, scattered at posterior lens capsule 51′ and correspondingly deflected as light beam 15′. Light beam 15′ strikes beam splitter 21 of detection system 20 at a position X, and does not reach detection device 35.

As previously in FIGS. 3a and 3b, eyes 50 in FIGS. 4a and 4b are depicted with and without the presence of a lens body of a lens 51.

Surgical microscope system 100 of the embodiment depicted in FIG. 2 can advantageously have associated with it a treatment unit 40 that encompasses, for example, a laser 43, preferably a short-pulse laser such as a femtosecond laser 43. Laser light can be coupled into the eye by means of this laser 43, with the result that automated treatment methods such the disintegration of lens 51 of the eye, or other treatment methods, can be carried out. Surgical microscope system 100 thereby becomes an automatable treatment system for treating cataracts.

As presented in conjunction with FIGS. 4a and 4b, light that travels into specific regions of lens 51 of the eye and/or of posterior lens capsule 51′ is scattered in such a way that it experiences an apparent attenuation. The detection unit therefore makes it possible to sense the presence of turbidity by way of an evaluation of that attenuation. An additional evaluation capability is provided, as mentioned, by way of the first scattering upon irradiation into eye 50.

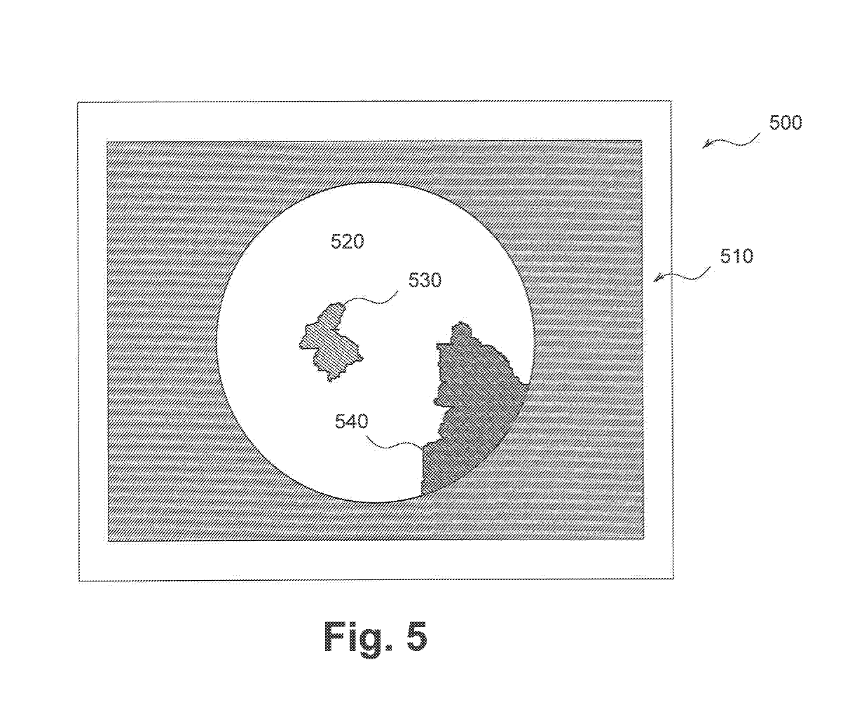

FIG. 5 depicts an image 500, obtainable by means of detection unit 25, that can be superimposed onto a microscopic image, for example using a superimposition device 33 of a viewing unit 30 of a surgical microscope system.

An image background 510 and an observation region 520 are visible in image 500. Observation region 520 corresponds, for example, to a pupil of eye 50 that is being investigated and/or treated. Background 510 corresponds to the anatomical regions of eye 50 that surround the pupil, for example to the iris. Within observation region 520, a scattering pattern having regions 530 and 540 of scattering that is of different intensity or is differently evaluated is evident. These correspond, for example, to regions where turbidity still exists as a result of membranes.

The scattering pattern was ascertained, for example, by means of a calculation unit 26 from an image determined by means of detection device 25. When an image 500 of this kind is correlated into a microscopic image of a viewing unit 30 via a superimposition device 33, i.e. superimposed identically in terms of location and magnification (in other words, congruently), the treating surgeon has the capability of performing a corresponding post-treatment in regions 530 and 540. This makes possible safe and reliable intra- and post-operative monitoring, while still in the operating room, of the complete removal of lens turbidity and/or turbidity of the posterior lens capsule.

### PARTS LIST

10 Illumination unit
11 Light source
12 Lens
13 Filter
15 Illumination light
15a, b, c Light rays
15′ Scattered light
20 Detection unit
21 Beam splitter
22 Light trap
23 Filter
24 Collimator lens
25 Detection device
26 Calculation unit
27 Output
28 Display system
29 Output
30 Viewing unit
31 Beam splitter
32 Zoom system
33 Superimposition device
34 Tube
35 Eyepiece
40 Treatment unit
41 Optical element
42 Objective
43 Laser
44 Additional lens
50 Eye
51 Lens of eye
51′ Posterior lens capsule
53 Vitreous body
54 Retina
100 Surgical microscope system
510 Image background
520 Observation region
530, 540 Scattering regions
F Imaging point
X Impact point

What is claimed is:

1. A surgical microscope system (100) for cataract surgery comprising:
an illumination unit (10), the surgical microscope system (100) being configured to irradiate illumination light (15) of the illumination unit (10) into an eye (50);
a viewing unit (30) through which a microscopic image of the eye (50) can be viewed; and
a detection unit (20) by which a scattering (15′) of the illumination light (15) irradiated into the eye (50) is determinable, in the form of at least one of a scattering pattern and a scattering coefficient, by sensing a portion of the illumination light (15) radiated back by the eye (50).

2. The surgical microscope system (100) according to claim 1, wherein the surgical microscope system (100) is configured to irradiate illumination light (15) of a first polarization extent, and to sense light of a second polarization extent.

3. The surgical microscope system (100) according to claim 2, wherein the illumination unit (10) comprises a first polarizing filter (13) and/or wherein the detection unit (20) comprises a second polarizing filter (23).

4. The surgical microscope system (100) according to claim 1, wherein the detection unit (20) comprises a beam splitter (21) for outcoupling at least a portion of the illumination light (15) radiated back by the object (50).

5. The surgical microscope system (100) according to claim 1, wherein the detection unit (20) comprises at least one optical unit (23, 24), at least one image sensing device (25), and at least one calculation unit (26) in order to determine the scattering pattern and/or the scattering coefficient.

6. The surgical microscope system (100) according to claim 1, wherein the detection unit includes at least one output (27, 29) for outputting the scattering pattern and/or the scattering coefficient.

7. The surgical microscope system (100) according to claim 6, wherein the viewing unit (30) comprises a superimposition device (33), coupled to at least one of the outputs (27, 29) of the detection device (20), through which the scattering pattern made available by the detection unit (20) is superimposable onto the microscopic image viewable by means of the viewing unit (30).

8. The surgical microscope system (100) according to claim 1, further comprising a beam splitter (31) arranged to couple a portion of the illumination light (15) radiated back by the eye (50) into an observation beam path.

9. The surgical microscope system (100) claim 1, wherein the surgical microscope system (100) is configured to irradiate illumination light (15) in the red and/or infrared spectral range.

10. The surgical microscope system (100) according to claim 1, further comprising at least one treatment unit (40).

11. The surgical microscope system (100) according to claim 10, wherein the treatment unit (40) includes a short-pulse laser, wherein a laser beam generated by the short-pulse laser (43) being irradiatable into the eye (50).

12. The surgical microscope system (100) according to claim 11, wherein the short-pulse laser is a nanosecond laser or a femtosecond laser (43).
